SEQUENCE LISTING (3 sequences)

SEQ ID NO: 1 - protein, 60 aa, Homo sapiens
KAVLTIDEKGTEAAGAMFLEAIPMSIPPEVKFNKPFVFLMIEQNTKSPLFMGKVVNPTQK

SEQ ID NO: 2 - protein, 30 aa, Homo sapiens
  misc_feature (2)..(2): Xaa can be any naturally occurring amino acid
MXIPPEVKFNKPFVFLMIDQNTKVPLFMGK

SEQ ID NO: 3 - protein, 28 aa, Homo sapiens
FNKPFVFLMIEQNTKSPLFMGKVVNPTQ

(19) United States
(12) Patent Application Publication
(10) Pub. No.: US 2023/0158108 A1
(43) Pub. Date: May 25, 2023

(54) IMMUNOTHROMBOSIS IN COVID-19 ACUTE RESPIRATORY DISTRESS SYNDROME

(71) Applicant: University of Utah Research Foundation, Salt Lake City, UT (US)

(72) Inventors: Christian Con Yost, Salt Lake City, UT (US); Joshua D. Schiffman, Salt Lake City, UT (US)

(21) Appl. No.: 17/923,180

(22) PCT Filed: May 4, 2021
(86) PCT No.: PCT/US2021/030685

§ 371 Date: Nov. 3, 2022

Related U.S. Application Data
(60) Provisional application No. 63/019,819, filed on May 4, 2020.

(51) Int. Cl. A61K 38/17; A61P 29/00

(52) U.S. Cl. CPC A61K 38/1709; A61P 29/00

(57) ABSTRACT

Disclosed are methods for treating neutrophil extracellular trap (NET) mediated inflammatory tissue damage in a patient having COVID-19, comprising administering to the patient having COVID-19 an effective amount of a pharmaceutical composition comprising a NET-inhibitory peptide (NIP) and a pharmaceutically acceptable carrier to reduce a pathological effect or symptom of the NET-mediated inflammatory tissue damage Disclosed are methods for inhibiting NET formation in a patient having COVID-19 comprising administering to the patient having COVID-19 an effective amount of a pharmaceutical composition comprising a NIP and a pharmaceutically acceptable carrier to substantially inhibit NET formation.

### CROSS-REFERENCE TO RELATED APPLICATIONS

This application claims the benefit of U.S. Provisional Patent Application No. 63/019,819, filed on May 4, 2020, which is incorporated by reference herein in its entirety.

### STATEMENT REGARDING FEDERALLY SPONSORED RESEARCH

This invention was made with government support under Grant Numbers R01093826, 5P30CA045508-30, R01HL135265, K01AG059892, R35HL145237, R01HL142804, R61HL141783 awarded by the National Institutes of Health. The government has certain rights in this invention.

### BACKGROUND

SARS-CoV-2 infection is now confirmed in over 3.5 million individuals worldwide and COVID-19 has caused nearly 250,000 fatalities globally. As the pandemic continues, evidence suggests that microthrombi and coagulopathy contribute to COVID-19 pathogenicity. Up to 10% of all COVID-19 patients will become critically ill with multiorgan failure and require Intensive Care Unit (ICU) admission, putting an unprecedented strain on healthcare systems. Successfully meeting the challenge of COVID-19 will require new insights into disease pathogenesis and novel treatments for patients. The interaction of neutrophil extracellular traps (NETs) with activated platelets, known as immunothrombosis, may explain the thrombotic events described in many COVID-19 patients. Thus, inhibiting NET formation in COVID-19 could ameliorate NET-mediated inflammatory and thrombotic tissue damage associated with COVID-19 acute respiratory distress syndrome (ARDS) and death.

Polymorphonuclear leukocytes (PMNs; neutrophils) produce NETs through a regulated cell death process termed NETosis. NETs are extracellular 3-dimensional lattices of decondensed chromatin decorated with histones and antimicrobial proteins released upon stimulation. Pathogens, including respiratory viruses, induce NETosis leading to NETs which physically trap and kill microbes as part of the innate immune system. When triggered by activated platelets, NETosis can become dysregulated leading to NET-mediated tissue damage, hypercoagulability, and thrombosis associated with both acute and chronic inflammatory disease. The contribution of NETosis to acute sepsis and ARDS pathogenesis is well documented, with NETs causing vascular tissue damage and scattered microthrombi leading to multiorgan failure and death.

### BRIEF SUMMARY

The most common complications of COVID-19 include ARDS, sepsis, and multiorgan dysfunction syndrome associated with high morbidity and mortality rates. Similar to other infectious syndromes, the tissue injury seen in SARS-CoV-2 infection is driven by dysregulated immune response, release of inflammatory cytokines, and development of pathogenic microvascular thrombi. Disclosed herein is clinical evidence that increased NETosis associates with COVID-19-related ARDS and is a potential biomarker for disease severity is provided. COVID-19 patients have increased plasma platelet factor 4 (PF4) and RANTES, which are known to trigger NETosis and immunothrombosis. Neonatal NET-Inhibitory Factor (nNIF), a NET-inhibitory peptide discovered in human umbilical cord blood, inhibits NET formation induced by plasma from COVID-19 patients.

Disclosed are methods for treating neutrophil extracellular trap (NET) mediated inflammatory tissue damage in a patient having a corona virus infection or disease resulting from a corona virus infection, comprising administering to the patient having a corona virus infection or disease resulting from a corona virus infection an effective amount of a pharmaceutical composition comprising a NET-inhibitory peptide (NIP) and a pharmaceutically acceptable carrier to reduce a pathological effect or symptom of the NET-mediated inflammatory tissue damage.

Disclosed are methods for inhibiting NET formation in a patient having a corona virus infection or disease resulting from a corona virus infection comprising administering to the patient having a corona virus infection or disease resulting from a corona virus infection an effective amount of a pharmaceutical composition comprising a NIP and a pharmaceutically acceptable carrier to substantially inhibit NET formation.

Disclosed are methods for treating neutrophil extracellular trap (NET) mediated inflammatory tissue damage and/or NET mediated microthrombi in a patient infected with SARS-CoV-2, comprising: administering to the patient infected with SARS-CoV-2 an effective amount of a pharmaceutical composition comprising a NET-inhibitory peptide (NIP) and a pharmaceutically acceptable carrier to reduce a pathological effect or symptom of the NET-mediated inflammatory tissue damage and/or to reduce NET mediated microthrombi in the patient.

Disclosed are methods for inhibiting neutrophil extracellular trap (NET) formation in a patient infected with SARS-CoV-2 comprising: administering to the patient infected with SARS-CoV-2 an effective amount of a pharmaceutical composition comprising a NET-inhibitory peptide (NIP) and a pharmaceutically acceptable carrier to substantially inhibit NET formation.

Disclosed are methods for inhibiting NETosis induced by soluble factors in plasma in a patient infected with SARS-CoV-2 comprising: administering to the patient infected with SARS-CoV-2 an effective amount of a pharmaceutical composition comprising a NET-inhibitory peptide (NIP) and a pharmaceutically acceptable carrier thereby inhibiting NETosis induced by soluble factors in plasma in a patient infected with SARS-CoV-2.

Disclosed are methods for preventing neutrophil extracellular trap (NET) mediated inflammatory tissue damage and/or NET mediated microthrombi in a patient infected with SARS-CoV-2, comprising: administering to the patient infected with SARS-CoV-2 an effective amount of a pharmaceutical composition comprising a NET-inhibitory peptide (NIP) and a pharmaceutically acceptable carrier to reduce a pathological effect or symptom of the NET-mediated inflammatory tissue damage and/or to reduce NET mediated microthrombi.

Disclosed are methods for inhibiting NETosis induced by soluble factors in plasma in a patient infected with SARS-CoV-2 comprising: administering to the patient infected with SARS-CoV-2 an effective amount of a pharmaceutical composition comprising a NET-inhibitory peptide (NIP) and a pharmaceutically acceptable carrier thereby inhibiting NETosis induced by soluble factors in plasma in a patient infected with SARS-CoV-2.

Disclosed are methods for preventing neutrophil extracellular trap (NET) mediated inflammatory tissue damage and/or NET mediated microthrombi in a patient infected with SARS-CoV-2, comprising: administering to the patient infected with SARS-CoV-2 an effective amount of a pharmaceutical composition comprising a NET-inhibitory peptide (NIP) and a pharmaceutically acceptable carrier to reduce a pathological effect or symptom of the NET-mediated inflammatory tissue damage and/or to reduce NET mediated microthrombi.

Disclosed are methods for preventing plasma from a SARS-CoV-2 infected patient from inducing NET formation in the patient comprising: administering to a patient an effective amount of a pharmaceutical composition comprising a NET-inhibitory peptide (NIP) and a pharmaceutically acceptable carrier to reduce NET formation from neutrophils induced by the plasma.

Disclosed are methods of reducing one or more platelet activation factors in a patient infected with SARS-CoV-2 comprising: administering to the patient infected with SARS-CoV-2 an effective amount of a pharmaceutical composition comprising a NET-inhibitory peptide (NIP) and a pharmaceutically acceptable carrier to substantially reduce the one or more platelet activation factors.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1I show increased plasma NETosis is related to increased COVID-19 severity. (FIG. 1A) Neutrophils and NETosis were stained for using immunofluorescence in lung tissue obtained at autopsy from COVID-19 patients (n=3). Commercially available normal lung tissue was stained as a negative control. Neutrophils express MPO (red) and NET-forming neutrophils express citrullinated histone-H3 (green). DAPI served as a nuclear DNA counterstain (blue). Cyan fluorescence represents the colocalization of citrullinated histone H3 with DNA. The yellow arrows point to neutrophils not undergoing NETosis and the white arrows to neutrophils undergoing NETosis. The dashed line highlights a thrombus in the microvasculature. The scale bars indicate 100 μm. (FIG. 1B-FIG. 1E) MPO-DNA ELISA was used to assess NETosis in plasma and tracheal aspirate samples from patients in our COVID-19 prospective cohort and in age- and sex-matched healthy donors; each colored dot represents an individual participant: gray (healthy control), blue (non-intubated COVID-19), red (intubated COVID-19), and green (convalescent COVID-19). (FIG. 1B) plasma NETosis levels were compared across all groups: healthy adult donors (n=14), adults hospitalized with COVID-19 but not intubated for ARDS (n=17), adults intubated for COVID-19 ARDS (n=5), and adults recovered from COVID-19 (n=2). The y-axis depicts plasma NETosis expressed as percent control±SEM. (FIG. 1C) Plasma NETosis levels were compared in 2 groups of COVID-19 patients: Survivors (n=19) vs. Non-survivors (n=3). The y-axis depicts plasma NETosis expressed as percent control±SEM. The One-way ANOVA statistical tool was employed with Tukey's multiple comparisons post hoc testing. (FIG. 1D) Plasma NETosis levels were correlated with the PaO2/FiO2 ratio measure of respiratory failure for all hospitalized COVID-19 patients (n=22). The Spearman's rank-correlation statistical tool was used. (FIG. 1E) Plasma NETosis levels in adult COVID-19 patients (n=22) were compared with NETosis levels quantified in the tracheal aspirate samples of intubated COVID-19 patients (n=3). The y-axis depicts NETosis levels expressed as percent control±SEM. The dashed line denotes the mean healthy donor plasma NETosis level. (FIG. 1F) Confocal microscopy was used to assess NET formation qualitatively in PMNs isolated from healthy donors (n=10) incubated without poly (I:C) (“Control”) vs. with poly I:C (1 μg/mL) for 2 hours (“Poly I:C”). Representative images show NETs (magenta; white arrows) and nuclear DNA (green). The scale bars denote 20 μm. (FIG. 1G) A high throughput cell-free DNA fluorescence assay was used to quantify NET formation in PMNs from healthy adults treated as in (FIG. 1F). The y-axis depicts NETosis measured as fold change over baseline relative fluorescence units±SEM. PMNs treated with vehicle control serve as the baseline PMN NETosis level, which was arbitrarily set at 1. The One-way ANOVA statistical tool was employed with Tukey's multiple comparisons post hoc testing. (FIG. 1H) Confocal microscopy was used to assess NET formation qualitatively in PMNs isolated from COVID-19 patients (n=8) incubated with and without poly (I:C) per (FIG. 1F). Representative images (n=8) show NETs (magenta; white arrows) and nuclear DNA (green). The scale bars denote 20 μm. (FIG. 10 High throughput cell-free DNA fluorescence assay was used to quantify NET formation in PMNs from COVID-19 patients treated as in (H). The y-axis depicts NETosis measured as fold change over baseline relative fluorescence units±SEM. PMNs treated with vehicle control serve as the baseline PMN NETosis level, which was arbitrarily set at 1. The One-way ANOVA statistical tool was employed with Tukey's multiple comparisons post hoc testing.

FIGS. 2A-2F show NETosis correlates with microthrombi formation and platelet deposition in COVID-19 patients. (FIG. 2A) NETosis and activated platelets were stained using immunofluorescence in COVID-19 lung tissue obtained at autopsy (n=3). NET forming neutrophils (MPO, white) express citrullinated histone-H3 (green). Microthrombi stained for both NETs and platelets (PF4; red). DAPI served as a nuclear DNA counterstain (blue). Yellow arrows point to thrombi with NETs in cases #1 and #3. Colocalized PF4 and NETs were not present in the analyzed lung sample from case #2. The scale bars indicate 100 μm. (FIG. 2B) Flow cytometry was used to quantify platelet-neutrophil aggregates in whole blood from COVID-19 patients (n=5) and healthy donors (n=6). The student's t-test statistical tool was used. ELISA was used to quantify plasma levels of (FIG. 2C) D-dimer, (FIG. 2D) VWF antigen, (FIG. 2E) PF4, and (FIG. 2F) RANTES in 18-22 COVID-19 patients and 5-7 healthy donors. The Mann-Whitney or student's t-test statistical tools were used depending on the normality of distribution. Grey dots represent healthy donors, and red circled dots represent COVID-19 patients.

FIGS. 3A-3B show nNIF blocks NETosis induced by soluble factors in plasma from COVID-19 patients. (FIG. 3A) Confocal microscopy was used to assess NET formation qualitatively in PMNs isolated from healthy adults (n=4) incubated with plasma samples from 16 different COVID-19 patients. The PMNs were incubated with either autologous plasma from the healthy adult donor or COVID-19 patient plasma and assessed for NET formation after a 2-hour incubation. PMNs were preincubated for 1 hour with either nNIF (1 nM) or its inactive, scrambled peptide control (SCR; 1 nM). Representative images from each experimental arm show NETs (magenta; white arrows) and nuclear DNA (green). The scale bars denote 20 μm. (FIG. 3B) A high throughput cell-free DNA fluorescence assay was used to quantify NET formation in the PMNs treated in (FIG. 3A). The y-axis depicts NETosis measured as fold change over baseline relative fluorescence units±SEM. The PMNs treated with autologous healthy plasma serve as the baseline PMN NETosis level, arbitrarily set at 1. The One-way ANOVA statistical tool was employed with Tukey's multiple comparisons post hoc testing.

FIGS. 4A-4B show COVID-19 PMNs demonstrate increased NETosis at baseline. We assessed NETosis in unstimulated PMNs isolated from healthy adult donors (n=9) and COVID-19 patients (n=4) using live cell imaging and cell-free DNA quantitation. (FIG. 4A) Representative images show NETs (magenta; white arrows) and nuclear DNA (green). The scale bar denotes 20 μm. (FIG. 4B) NETosis was assessed using a fluorescence-based cell-free DNA assay as described in Methods. The y-axis depicts NETosis shown as relative cell-free DNA fluorescence±SEM. the student's t-test statistical tool was used.

FIG. 5 shows platelet-neutrophil aggregates are elevated in COVID-19 patient plasma compared to that of healthy donors. Representative flow cytometry scatterplots showing cell gating and dual staining for platelets (CD41) and PMNs (CD66b), see FIG. 2B. Red coloring denotes flow cytometric gating on PMNs.

FIG. 6 shows differential NETosis induction by COVID-19 patient plasma.

FIG. 7 shows ex vivo results showing NIP (e.g. nNIF) inhibition of COVID-19 plasma induced NETosis.

FIG. 8 shows a comparison of treatment with dexamethasone and nNIF.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a peptide” includes a plurality of such peptides, reference to “the composition” is a reference to one or more compositions and equivalents thereof known to those skilled in the art, and so forth.

As used herein, the term “subject” or “patient” can be used interchangeably and refer to any organism to which a composition of this invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates; and the like.

A “NET-Inhibitory Peptide (NIP)” is an anti-inflammatory agent that inhibits neutrophil extracellular trap (NET) formation. Examples of NIPs include, but are not limited to: a neonatal NET-Inhibitory Factor (nNIF); a pharmaceutically acceptable salt of a nNIF; an analog of a naturally occurring form of nNIF, which nNIF analog inhibits NETosis and/or the formation of NETs and is structurally altered, relative to a given human nNIF, by at least one amino acid addition, deletion, substitution, or by incorporation of one or more amino acids with a blocking group; a pharmaceutically acceptable salt of a nNIF analog; a nNIF-Related Peptide (nNRP); a pharmaceutically acceptable salt of a nNRP; a nNRP analog; or a pharmaceutically acceptable salt of a nNRP analog. In some aspects, a nNIF analog or a nNRP analog is a mutant or variant form of nNIF or NRP, respectively.

A “neonatal Neutrophil Inhibitory Factor peptide” or “nNIF peptide” is defined herein as a nNIF which is naturally occurring in mammals or a variant thereof.

A “neonatal NIF-related peptide” or “nNRP” is defined herein as a Cancer-Associated SCM-Recognition, Immune Defense Suppression, and Serine Protease Protection Peptide (CRISPP) which is naturally occurring in humans of a variant thereof; A1ATm 358, which has been shown to inhibit NET formation; and other nNIF-related peptides; and as analogs of naturally occurring forms of nNRPs that inhibit NETosis and/or the formation of NETs and are structurally altered, relative to a given human nNRP, by at least one amino acid addition, deletion, substitution, or by incorporation of one or more amino acids with a blocking group.

As used herein an amino acid “substitution” refers to the replacement of one amino acid residue by a different amino acid residue. The substituted amino acid may be any of the 20 amino acids commonly found in human proteins, as well as atypical or non-naturally occurring amino acids. A substitution of an amino acid residue can be considered conservative or non-conservative. Conservative substitutions are those within the following groups: Ser, Thr, and Cys; Leu, ILe, and Val; Glu and Asp; Lys and Arg; Phe, Tyr, and Trp; and Gln, Asn, Glu, Asp, and His. In some aspects, the substitution can be a non-naturally occurring substitution. For example, the substitution may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH2(CH2)nCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline.

As used herein, the term “wild-type” refers to a gene or gene product which has the characteristics of that gene or gene product when isolated from a naturally-occurring source.

The terms “variant” and “mutant” are used interchangeably herein. As used herein, the term “variant” refers to a modified nucleic acid or protein which displays the same characteristics when compared to a reference nucleic acid or protein sequence. A variant can be at least 65, 70, 75, 80, 85, 90, 95, or 99 percent homologous to a reference sequence. In some aspects, a reference sequence can be a wild type NIP nucleic acid sequence or a wild type NIP protein sequence. A “variant,” “analog,” “variant thereof,” or “analog thereof” can mean a difference in some way from the reference sequence other than just a simple deletion of an N- and/or C-terminal amino acid residue or residues. Where the variant includes a substitution of an amino acid residue, the substitution can be considered conservative or non-conservative. Variants can include at least one substitution and/or at least one addition, there may also be at least one deletion. Variants can also include one or more non-naturally occurring residues.

The term “percent (%) homology” is used interchangeably herein with the term “percent (%) identity” and refers to the level of nucleic acid or amino acid sequence identity when aligned with a wild type sequence using a sequence alignment program. For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 80, 85, 90, 95, 98% or more sequence identity to a given sequence, e.g., the coding sequence for anyone of the inventive polypeptides, as described herein. Exemplary computer programs which can be used to determine identity between two sequences include, but are not limited to, the suite of BLAST programs, e.g., BLASTN, BLASTX, and TBLASTX, BLASTP and TBLASTN, publicly available on the Internet. See also, Altschul, et al., 1990 and Altschul, et al., 1997. Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62 matrix. (See, e.g., Altschul, S. F., et al., Nucleic Acids Res. 25:3389-3402, 1997.) A preferred alignment of selected sequences in order to determine“% identity” between two or more sequences, is performed using for example, the CLUSTAL-W program in Mac Vector version 13.0.7, operated with default parameters, including an open gap penalty of 10.0, an extended gap penalty of 0.1, and a BLOSUM 30 similarity matrix.

Substitutions, deletions, insertions or any combination thereof may be used to arrive at a final derivative, variant, or analog. Generally, these changes are done on a few nucleotides to minimize the alteration of the molecule. However, larger changes may be tolerated in certain circumstances.

Generally, the nucleotide identity between individual variant sequences can be at least 90%, 91%, 92%, 93%, 94%, 95%, 96%, 97%, 98%, 99%, or 100%. Thus, a “variant sequence” can be one with the specified identity to the parent or reference sequence (e.g. wild-type sequence) of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. For example, a “variant sequence” can be a sequence that contains 1, 2, or 3, 4 nucleotide base changes as compared to the parent or reference sequence of the invention, and shares or improves biological function, specificity and/or activity of the parent sequence. Thus, a “variant sequence” can be one with the specified identity to the parent sequence of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. The variant sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of a reference sequence (e.g. wild-type sequence, a nNIF nucleic acid sequence or nNIF protein sequence).

By an “effective amount” of a composition as provided herein is meant a sufficient amount of the composition to provide the desired effect. The exact amount required will vary from subject to subject, depending on the species, age, and general condition of the subject, the severity of disease (or underlying genetic defect) that is being treated, the particular composition used, its mode of administration, and the like. Thus, it is not possible to specify an exact “effective amount.” However, an appropriate “effective amount” may be determined by one of ordinary skill in the art using only routine experimentation. The term “therapeutically effective amount” means an amount of a therapeutic, prophylactic, and/or diagnostic agent (e.g., NET inhibitory peptide) that is sufficient, when administered to a subject suffering from or susceptible to infection with SARS-CoV-2/COVID-19, to treat, alleviate, ameliorate, relieve, alleviate symptoms of, prevent, delay onset of, inhibit progression of, reduce severity of, and/or reduce incidence of infection with SARS-CoV-2/COVID-19 (e.g. inhibiting NETosis, inhibiting, preventing or reducing neutrophil extracellular trap (NET) mediated inflammatory tissue damage and/or NET mediated microthrombi, or reducing one or more platelet activation factors).

By “treat” is meant to administer a peptide or composition of the invention to a subject, such as a human or other mammal (for example, an animal model), that has an increased susceptibility for developing infection with SARS-CoV-2/COVID-19 or that has an infection with SARS-CoV-2/COVID-19, in order to prevent or delay a worsening of the effects of the disease or condition, or to partially or fully reverse the effects of the disease or condition (e.g. inhibiting NETosis, inhibiting, preventing or reducing neutrophil extracellular trap (NET) mediated inflammatory tissue damage and/or NET mediated microthrombi, or reducing one or more platelet activation factors).

By “prevent” is meant to minimize the chance that a subject who has an increased susceptibility for developing an infection with SARS-CoV-2/COVID-19 actually develops the infection or disease or otherwise develops a cause of symptom thereof (e.g. inhibiting NETosis, inhibiting, preventing or reducing neutrophil extracellular trap (NET) mediated inflammatory tissue damage and/or NET mediated microthrombi, or reducing one or more platelet activation factors).

As used herein, the terms “administering” and “administration” refer to any method of providing a disclosed peptide, composition, or a pharmaceutical preparation to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration for a disclosed composition or a disclosed peptide so as to treat a subject or inhibit NET formation. In an aspect, the skilled person can also alter or modify an aspect of an administering step so as to improve efficacy of a disclosed peptide, composition, or a pharmaceutical preparation.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Peptides

Disclosed herein are NET-inhibitory peptides (NIPs). In some aspects, NIPs can be, but are not limited to a neonatal NET-Inhibitory Factor (nNIF), a nNIF-Related Peptide (nNRP), a pharmaceutically acceptable salt of a nNIF or nNRP, or variants thereof.

NIPs, such as nNIFs, and/or nNRPs can be generated using standard techniques of peptide chemistry and can be assessed for inhibition of NETosis and/or NET formation activity, all according to the guidance provided herein. In some aspects, the disclosed peptides can be the sequences of human nNIF (SEQ ID NO:1) and/or CRISPP (SEQ ID NO:2), as follows (wherein X can be any naturally occurring amino acid):

(SEQ ID NO: 1)
KAVLTIDEKGTEAAGAMFLEAIPMSIPPEV
KFNKPFVFLMIEQNTKSPLFMGKVVNPTQK
(SEQ ID NO: 2)
MXIPPEVKFNKPFVFLMIDQNTKVPLFMGK

In some aspects, the disclosed peptides can be variants of the sequences of human nNIF (SEQ ID NO:1) and/or CRISPP (SEQ ID NO:2).

Any substitution, addition, or deletion of an amino acid or amino acids of a NIP, nNIF, and/or nNRP that does not destroy the NET-inhibitory activity of the NIP, nNIF, and/or nNRP is disclosed. In certain embodiments, the NIP, nNIF, and/or nNRP analogs/variants are at least as active as the native human NIP, nNIF, and/or nNRP. NET-inhibitory activity may be determined in vitro as described in this disclosure. In other embodiments, the NIP, nNIF, and/or nNRP analog/variant has one or more enhanced properties compared with the native human NIP, nNIF, and/or nNRP. For example, such analogs may exhibit enhanced serum stability, enhanced receptor binding and enhanced signal-transducing activity. Other modifications to NIPs, nNIFs, nNIF analogs, nNRPs, and/or nNRP analogs that may usefully be employed in this disclosure are those which render the molecule resistant to oxidation.

In some aspects, the peptide can be a portion of SEQ ID NO:1 consisting of the sequence FNKPFVFLMIEQNTKSPLFMGKVVNPTQ (SEQ ID NO:3). Thus, in some aspects, the peptide comprises or consists of SEQ ID NO:3 or a variant thereof.

The disclosure also encompasses non-conservative substitutions of amino acids in any vertebrate NIP, nNIF, and/or nNRP sequence, provided that the non-conservative substitutions occur at amino acid positions known to vary in NIPs, nNIFs, and/or nNRPs isolated from different species. Non-conserved residue positions are readily determined by aligning known vertebrate NIP, nNIF, and/or nNRP sequences.

### C. Compositions

Disclosed are compositions comprising any of the disclosed peptides. Disclosed are compositions comprising one or more of the disclosed NIPs or variants thereof.

In some aspects, the composition can be a pharmaceutical composition (e.g., formulation, preparation, medicament) comprising, or consisting essentially of, or consisting of as an active ingredient, a nucleic acid construct, vector, protein or recombinant cell as described herein, and a pharmaceutically acceptable carrier, diluent, or excipient.

Disclosed are composition and formulations of the disclosed peptides with a pharmaceutically acceptable carrier or diluent. For example, disclosed are pharmaceutical compositions, comprising the peptides disclosed herein, and a pharmaceutically acceptable carrier.

For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, Pa. 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. In the methods described herein, delivery of the disclosed compositions to cells can be via a variety of mechanisms. The pharmaceutical composition may be administered in a number of ways depending on whether local or systemic treatment is desired, and on the area to be treated.

1. Delivery of Compositions

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

### D. Methods

Disclosed are methods for treating neutrophil extracellular trap (NET) mediated inflammatory tissue damage in a patient having a corona virus infection or disease resulting from a corona virus infection, comprising administering to the patient having a corona virus infection or disease resulting from a corona virus infection an effective amount of a pharmaceutical composition comprising a NET-inhibitory peptide (NIP) and a pharmaceutically acceptable carrier to reduce a pathological effect or symptom of the NET-mediated inflammatory tissue damage.

Disclosed are methods for inhibiting NET formation in a patient having a corona virus infection or disease resulting from a corona virus infection comprising administering to the patient having a corona virus infection or disease resulting from a corona virus infection an effective amount of a pharmaceutical composition comprising a NIP and a pharmaceutically acceptable carrier to substantially inhibit NET formation.

In some aspects, a corona virus infection can be from an alphacoronavirus, betacoronaviruses, gammacoronavirus, or deltacoronaviruses. Examples of alphacoronaviruses can include, but are not limited to, Alphacoronavirus 1, Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512. Examples of betacoronaviruses can include, but are not limited to, Betacoronavirus 1 (Bovine Coronavirus, Human coronavirus OC43), Hedgehog coronavirus 1, Human coronavirus HKU1, Middle East respiratory syndrome-related coronavirus, Murine coronavirus, Pipistrellus bat coronavirus HKU5, Rousettus bat coronavirus HKU9, Severe acute respiratory syndrome-related coronavirus (SARS-CoV, SARS-CoV-2), Tylonycteris bat coronavirus HKU4. Examples of gammacoronaviruses can include, but are not limited to, Avian coronavirus, Beluga whale coronavirus SW1. Examples of deltacoronaviruses can include, but are not limited to, Bulbul coronavirus HKU11, Porcine coronavirus HKU15.

Disclosed are methods for treating neutrophil extracellular trap (NET) mediated inflammatory tissue damage and/or NET mediated thrombi in a patient infected with SARS-CoV-2, comprising administering to the patient infected with SARS-CoV-2 an effective amount of a pharmaceutical composition comprising a NET-inhibitory peptide (NIP) and a pharmaceutically acceptable carrier to reduce a pathological effect or symptom of the NET-mediated inflammatory tissue damage.

Disclosed are methods for preventing NET mediated inflammatory tissue damage and/or NET mediated microthrombi in a patient infected with SARS-CoV-2, comprising administering to the patient infected with SARS-CoV-2 an effective amount of a pharmaceutical composition comprising a NET-inhibitory peptide (NIP) and a pharmaceutically acceptable carrier to reduce a pathological effect or symptom of the NET-mediated inflammatory tissue damage and/or to reduce NET mediated microthrombi.

Disclosed are methods for inhibiting NET formation in a patient infected with SARS-CoV-2 comprising administering to the patient infected with SARS-CoV-2 an effective amount of a pharmaceutical composition comprising a NET-inhibitory peptide (NIP) and a pharmaceutically acceptable carrier to substantially inhibit NET formation.

Disclosed are methods for inhibiting NETosis induced by soluble factors in plasma in a patient infected with SARS-CoV-2 comprising administering to the patient infected with SARS-CoV-2 an effective amount of a pharmaceutical composition comprising a NIP and a pharmaceutically acceptable carrier thereby inhibiting NETosis induced by soluble factors in plasma in a patient infected with SARS-CoV-2. In some aspects, the soluble factors in the plasma of a patient infected with SARS-CoV-2 can be, but are not limited to, RANTES, platelet factor 4 (PF4), von willibrand factor (VWF), or P-selectin.

Disclosed are methods of reducing one or more platelet activation factors in a patient infected with SARS-CoV-2 comprising administering to the patient infected with SARS-CoV-2 an effective amount of a pharmaceutical composition comprising a NIP and a pharmaceutically acceptable carrier to substantially reduce the one or more platelet activation factors. In some aspects, the one or more platelet activation factors are RANTES, platelet factor 4, von willibrand factor, or P-selectin.

Disclosed are methods for reducing the level of soluble factors in plasma in a patient infected with SARS-CoV-2 inhibiting comprising administering to the patient infected with SARS-CoV-2 an effective amount of a pharmaceutical composition comprising a NIP and a pharmaceutically acceptable carrier to reduce the level of the soluble factors in the plasma of the patient infected. In some aspects, the soluble factors are platelet activation factors such as RANTES, platelet factor 4, von willibrand factor, or P-selectin. In some aspects, a patient infected with SARS-CoV-2 has COVID-19. In some aspects, a patient having COVID-19 has symptoms of the disease (e.g. NETosis, neutrophil extracellular trap (NET) mediated inflammatory tissue damage, NET mediated microthrombi, or an increase in one or more platelet activation factors such as RANTES, platelet factor 4, von willibrand factor, or P-selectin.

In some aspects, prior to administering a NIP to a patient, the patient is first identified or diagnosed as being SARS-CoV-2 positive. In some aspects, a patient having COVID-19 is a patient previously determined to be infected with SARS-CoV-2. In some aspects, a patient infected with SARS-CoV-2 is a patient with no symptoms, mild symptoms or severe symptoms of COVID-19.

In some aspects of the disclosed methods, the NIP is a nNIF or nNRP. In some aspects, the NIP is a peptide comprising the amino acid sequence of SEQ ID NO:1, SEQ ID NO:2 or SEQ ID NO:3. In some aspects the NIP is administered with a pharmaceutically acceptable carrier. In some aspects the NIP is administered in an amount sufficient to reduce a pathological effect or symptom of the NET-mediated inflammatory tissue damage and/or substantially inhibit NET formation. In some aspects, the NIP is a peptide consisting of the amino acid sequence of SEQ ID NO:1, SEQ ID NO:2 or SEQ ID NO:3. In some aspects, the NIP is a peptide consisting of the amino acid sequence of SEQ ID NO:1, SEQ ID NO:2 or SEQ ID NO:3 and is administered with a pharmaceutically acceptable carrier. In some aspects the NIP is administered in an amount sufficient to reduce a pathological effect or symptom of the NET-mediated inflammatory tissue damage and/or substantially inhibit NET formation.

In some aspects, the thrombi can be microthrombi. In some aspects, the thrombi can be venous or arterial, and occur anywhere in the body.

In some aspects, NIPs, such as nNIF or CRISPP, can be effective at preventing NET-mediated symptoms even prior to presentation to the hospital. In some aspects, NIPs can be used as a prophylactic before symptoms get worse.

In some aspects, the disclosed methods further include the step of first diagnosing a patient as having COVID-19. In some aspects, the step of diagnosing a patient as having COVID-19 comprises detecting the presence and/or levels of NETosis in the patient. In some aspects, increased levels of NETosis in a patient corresponds with increased COVID-19 severity and/or progression. In some aspects, NETosis can be a biomarker for COVID-19 severity and/or progression. In some aspects, the presence and/or levels of NETosis can be detected in blood, plasma, or tissue samples of a subject/patient. Based on the levels of NETosis, a patient can be identified as a responder to NIP treatment. For example, the higher the levels of NETosis, the more likely a patient is to be successfully treated with a NIP, such as nNIF or NRP. In some aspects, the dose of a NIP can be adjusted based on the levels of NETosis.

In some aspects, the NET mediated inflammatory tissue damage and/or NET mediated thrombi (e.g. microthrombi), and/or NET formation, and/or NETosis is reduced (or inhibited) in an effective amount. In some aspects, the NET mediated inflammatory tissue damage and/or NET mediated microthrombi, and/or NET formation, and/or NETosis is reduced by 5, 10, 15, 20, 25, 30, 35, 40, 45, 50, 55, 60, 65, 70, 75, 80, 85, 90, 95 or 100%. In some aspects, the NET mediated inflammatory tissue damage and/or NET mediated microthrombi, and/or NET formation, and/or NETosis is reduced by 1-90%, 1-50%, 2-75%, 2-40%, 3-60%, 3-30%, 4-50%, 4-25%, 5-50%, or 5-20%.

In some aspects, NET mediated inflammatory tissue damage and/or NET mediated thrombi, such as microthrombi, and/or NET formation, and/or NETosis is reduced (or inhibited), for example, by 5-50%, when compared to a sample from the patient infected with SARS-CoV-2 that did not receive an effective amount of the pharmaceutical composition comprising a NIP and a pharmaceutically acceptable carrier. In some aspects, NET mediated inflammatory tissue damage and/or NET mediated microthrombi, and/or NET formation, and/or NETosis is reduced, for example, by 5-50%, when compared to a reference or control. In some aspects, a reference or control can be a patient infected with SARS-CoV-2 that did not receive an effective amount of the pharmaceutical composition comprising a NIP, a patient infected with SARS-CoV-2 that received a scrambled NIP, or a patient infected with SARS-CoV-2 that received a solution that does not contain NIP, such as a solution that only contains the pharmaceutically acceptable carrier (e.g. saline).

Disclosed are methods of reducing one or more platelet activation factors in a patient infected with SARS-CoV-2 comprising administering to the patient infected with SARS-CoV-2 an effective amount of a pharmaceutical composition comprising a NIP and a pharmaceutically acceptable carrier to substantially reduce the one or more platelet activation factors. In some aspects, the one or more platelet activation factors are RANTES, platelet factor 4, von willibrand factor, or P-selectin.

In some aspects, the patient in any of the disclosed methods administered a NIP has medium or high inducing plasma. In some aspects, patients can be stratified into low (NETosis) inducing plasma, medium (NETosis) inducing plasma or high (NETosis) inducing plasma. In some aspects, prior to administering the effective amount of a pharmaceutical composition comprising a NIP and a pharmaceutically acceptable carrier, the patient is identified as having medium or high inducing plasma. As described herein, low, medium, or high inducing plasma classifications refers to the level of NETosis induction relative to neutrophils from a healthy subject. For example, low inducing plasma can be a 1.2-2 fold increase over healthy neutrophil NETosis induction; medium inducing plasma can be a 2-3 fold increase over healthy neutrophil NETosis induction, high inducing plasma can be a greater than 3 fold increase over healthy neutrophil NETosis induction. In some aspects, a patient with no inducing plasma can be plasma with a less than 1.2 fold increase over healthy neutrophil NETosis induction.

In some aspects, a patient has medium or high inducing plasma and the NET mediated inflammatory tissue damage and/or NET mediated microthrombi, and/or NETosis formation is reduced or inhibited by 5-50% as compared to a patient with low inducing plasma. In some aspects, a patient has medium or high inducing plasma and the NET formation is reduced or inhibited by 5-50% as compared to a patient with low or no inducing plasma. In some aspects, a patient has medium or high inducing plasma and NETosis induced by soluble factors in plasma is inhibited by 5-50% as compared to a patient with no or low inducing plasma.

In some aspects, the subject can be stratified on one more characteristics and stratified for treatment. For example, in some aspects, a subject can be identified as having low inducing plasma, medium inducing plasma or high inducing plasma as described herein. Based on stratification, the efficacy of treatment with nNIF can be determined.

In some aspects, patients can be divided in quartiles. In some aspects, the disclosed methods involve comparing the level of NETosis induction for a subject with a predetermined value. The predetermined value can take a variety of forms. It can be single cut-off value, such as a median or mean. It can be established based upon comparative groups, such as, for example, where the amount of induction in one defined group is double the amount of induction in another defined group. It can be a range, for example, where the tested population is divided equally (or unequally) into groups, such as a low inducing group, a medium inducing group and a high inducing group, or into quartiles, the lowest quartile being subjects with the highest induction and the highest quartile being subjects with no induction, or into tertiles the lowest tertile being subjects with the highest induction and the highest tertile being subjects with the lowest induction. The predetermined value may be a cut-off value which is predetermined by the fact that a group having an induction level no less than the cut-off value demonstrates a statistically significant increase in the induction of NETosis as compared to a comparative group. In some aspects, the comparative group is a group having a lower level of induction or a group of healthy neutrophils with no induction.

In some aspects, disclosed methods can include comparing the level of a biomarker for a subject with a predetermined value of the biomarker. In some aspects, the biomarker can be a microthrombi biomarker. In some aspects, the biomarker can be platelet factor 4, RANTES, von willibrand factor, or P-selectin.

Also disclosed are combination therapies. In some aspects of the disclosed methods, the methods can further comprise administering one or more known standards of care for coronavirus infection. In some aspects, the one or more known standards of care for coronavirus infection are antibodies, nanobodies, antiviral small molecules, macromolecules of sulfated polysaccharides, or polypeptides. Frequent targets of the known standards of care are the viral spike protein, the host angiotensin converting enzyme 2, the host transmembrane protease serine 2, and clathrin-mediated endocytosis. For example, the disclosed methods comprising administering an NIP can further comprise administering the NIP in combination with one or more of remdesivir (Veklury), Nafamostat, Avigan (favilavir), bamlanivimab, Olumiant and Baricinix (baricitinib), hydroxychloroquine/chloroquine, Casirivimab and imdevimab (formerly REGN-COV2), PTC299, Leronlimab (PRO 140), Bamlanivimab (LY-CoV555), Lenzilumab, Ivermectin, RLF-100 (aviptadil), Metformin (Glucophage, Glumetza, Riomet), AT-527, Actemra (tocilizumab), Niclocide (niclosamide), Convalescent plasma, Pepcid (famotidine), Kaletra (lopinavir-ritonavir), Remicade (infliximab), AZD7442, AZD7442, CT-P59, Heparin (UF and LMW), VIR-7831 (GSK4182136), JS016, Kevzara (sarilumab), SACCOVID (CD24Fc), Humira (adalimumab), COVI-GUARD (STI-1499), Dexamethasone (Dextenza, Ozurdex, others), PB1046, Galidesivir, Bucillamine, PF-00835321 (PF-07304814), Eliquis (Apixaban), Takhzyro (lanadelumab), Hydrocortisone, Ilaris (canakinumab), Colchicine (Mitigare, Colcrys), BLD-2660, Avigan (favilavir/avifavir), Rhu-pGSN (gelsolin), MK-4482, TXA127, LAM-002A (apilimod dimesylate), DNL758 (SAR443122), INOpulse, ABX464, AdMSCs, Losmapimod, Mavrilimumab, or Calquence (acalabrutinib), quinoline-based antimalarials ((hydroxy)-chloroquine and others), RAAS modifiers (captopril, losartan, and others), statins (atorvastatin and simvastatin), guanidino-based serine protease inhibitors (camostat and nafamostat), antibacterials (macrolides, clindamycin, and doxycycline), antiparasitics (ivermectin and niclosamide), cardiovascular drugs (amiodarone, verapamil, and tranexamic acid), antipsychotics (chlorpromazine), antivirals (umifenovir and oseltamivir), DPP-4 inhibitors (linagliptin), JAK inhibitors (baricitinib and others), sulfated glycosaminoglycans (UFH and LMWHs) and polypeptides such as the enzymes DAS181 and rhACE2. Or monoclonal antibodies such as REGN10933 and REGN10987.

### EXAMPLES

### Example 1

### E. Methods

1. Study Design

In this investigator-initiated, prospective cohort study, patients admitted to the University of Utah Health Sciences Center with respiratory distress and positive SARS-CoV-2 testing were enrolled. Also enrolled were age- and sex-matched healthy adults as comparators for this study. Elements implicated in the pathogenesis of COVID-19 related sepsis, ARDS, and thrombosis, including NET formation and platelet activation were investigated.

2. Study Patients

60 mL of ACD-anticoagulated whole blood was collected from patients hospitalized with COVID-19. Enrollment criteria included age greater than 18, respiratory symptoms (cough, shortness of breath) or fever, hospital admission, positive SARS-CoV-2 testing, and informed consent. Demographic and illness severity data are summarized in Table 1.

TABLE 1
Clinical Characteristics of Healthy Donors and Hospitalized Patients with COVID-19

| | | | Hospitalized | | | | |
|---|---|---|---|---|---|---|---|
| | Healthy Donors | | Non-ICU COVID-19 | | ICU COVID-19 | | |
| | (n = 14) | | Infection (n = 15) | | Infection (n = 8) | | p Value |
| Age (mean, ±SD) | 50.6 | (±17.6) | 48.7 | (±13.9) | 63.4 | (±17.1) | 0.112 |

| Male (%) | 50% | 60% | | 62.5% | | 0.819 |
|---|---|---|---|---|---|---|
| Hispanic/Latino/Black (%) | 0% | 26.7% | | 37.5% | | 0.611 |
| BMI (mean, ±SD) | | 35.8 | (±9.6) | 31.6 | (±9.4) | 0.338 |
| Diabetes (%) | 0% | 40.0% | | 62.5% | | 0.326 |
| Hypertension (%) | 0% | 42.9% | | 50.0% | | 0.886 |
| Chronic lung disease (%) | 0% | 28.6% | | 25.0% | | 0.935 |
| SOFA score (mean, ±SD) | | 1.8 | (±1.0) | 5.8 | (±1.2) | <0.0001 |
| ARDS (%) | | 13.30% | | 100% | | <0.0001 |
| Mechanical Ventilation (%) | | 0.0% | | 62.5% | | 0.0001 |
| Survival to date (%) | | 100% | | 62.5% | | 0.0092 |
| WBC (mean, ±SD) | | 5.7 | (±1.9) | 8.5 | (±2.4) | 0.006 |
| Platelet count (mean, ±SD) | | 261 | (±112) | 264 | (±46) | 0.946 |

3. MPO-DNA ELISA

NETs were detected in plasma and tracheal aspirate samples using the MPO-DNA ELISA. An anti-human myeloperoxidase (MPO) primary antibody (Upstate Biotechnology) was used as the capture antibody and a peroxidase-labeled anti-DNA primary antibody (Cell Death Detection ELISA kit, Roche) was used as the detection antibody. Plasma and tracheal aspirate samples were diluted 1:3 and 1:15 with PBS, respectively. Results are reported as percent of control±SD.

4. PMN Isolation

PMNs were isolated from whole blood from healthy adults and COVID-19 patients using the EasySep Direct Human Neutrophil Isolation kit (Stemcell Technologies) with greater than 95% purity. The PMNs were resuspended to a concentration of 2×10⁶ cells/mL in serum free M-199 and performed all of the experiments at 37° C. in 5% CO₂/95% air.

5. NET Imaging and Quantification

Human NET formation was assessed. PMNs isolated from healthy adults and COVID-19 patients were treated with poly (I:C) (InVivoGen; 1 μg/mL; 2 hours) or COVID-19 patient plasma (undiluted; 2 hours) with and without a 1-hour pretreatment with nNIF or its inactive, scrambled peptide control (1 nM). For all experiments, NET formation was visualized using confocal microscopy (Olympus) with cell-permeable (SYTO Green, Molecular Probes) and cell-impermeable (SYTOX Orange, Molecular Probes) DNA fluorescent dyes. NET formation was quantitatively assessed using a cell-free DNA fluorescence assay with the SYTOX Green DNA dye. Relative fluorescence was quantified using a fluorometric plate reader with SoftMax software (Molecular Devices).

6. Immunofluorescence Staining for NETs and Platelets

Paraffin embedded autopsy specimens from three cases of COVID-19 from Weill Cornell Medical Center were stained largely as previously described. Commercially available healthy lung tissue served as a control (US Biomax [NCT086]). Briefly, to stain for neutrophils and NETs, we used mouse-anti-human MPO (1:400, MAB3174, R&D Systems) and rabbit-anti-human citrullinated Histone H3 (1:250, ab5103, Abcam). To stain for NETs and PF4, anti-citrullinated Histone H3 and mouse-anti-human PF4 (1:200sc-398979, Santa Cruz) were used with Alexa-488-conjugated anti-mouse and Alexa-568 conjugated anti-rabbit antibodies, blocked using a Mouse on Mouse Immunodetection kit (BMK-2202, Vector Laboratories), followed by incubation with anti-MPO antibodies and Alexa-647-conjugated anti-mouse antibody. We used DAPI as a nuclear counterstain (Life Technologies). Images were captured using confocal microscopy (Leica SP8) and analyzed using Volocity software (Version 6.3.0, PerkinElmer).

7. Plasma Coagulation Factor Assays

ELISA kits were used to quantify soluble PF4 and RANTES (R&D Systems) in plasma from COVID-19 patients and healthy donors. Additional ELISA kits (Abcam) were used to quantify VWF and D-dimers in the same samples.

8. Platelet-Neutrophil Aggregates

Whole blood was diluted 1:10 with Tyrodes/HEPES buffer and labeled platelets with anti-human CD41-APC antibodies (1:50, 559777, BD Biosciences) and PMNs with anti-human CD66b-V450 antibodies (1:20, 560861, BD Biosciences) for 15 minutes at 37° C. Fixation was performed with FACS lysis buffer and determined fluorescence on a CytoFlex Flow Cytometer (Becton-Dickinson).

9. Cytokine Array

Cytokine levels were measured in equal volumes of healthy donor and COVID-19 patient plasma using a multiplex bead array for IL8 and IL6 (Millipore, Billerica, Mass.) according to manufacturer's instructions and analyzed on a Luminex 200 machine.

10. nNIF Peptide Synthesis

nNIF and its inactive, scrambled peptide control were synthesized using standard techniques.

11. Statistical Analysis

For comparisons of the hospitalized patient groups, the mean±SD was determined. For each experimental variable, the mean±SEM was determined. For group comparisons an unpaired Student's t-test or a one-way ANOVA was used with Tukey's multiple comparisons post hoc testing as appropriate (GraphPad Prism, v8.4). Data not normally distributed were analyzed with a Mann-Whitney statistical test for pairwise comparisons. Correlations were calculated using Spearman's rank-correlation. All data used in each statistical test met the assumptions of the specific test. All tests were two-sided and P<0.05 was considered statistically significant.

### F. Results

1. Increased Plasma NETosis is Related to Increased COVID-19 Severity.

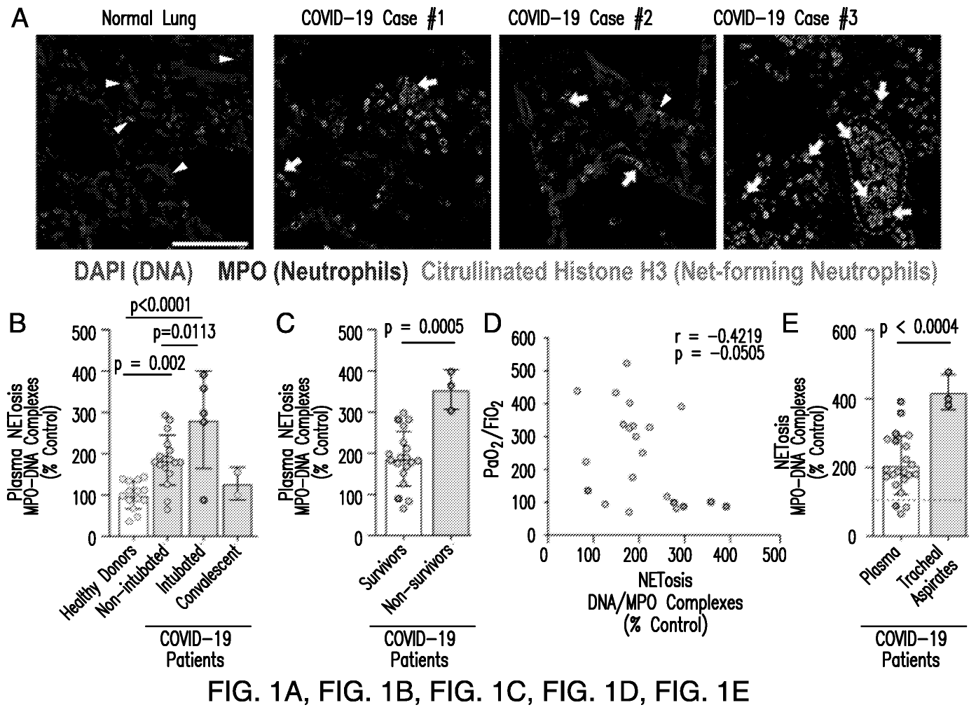

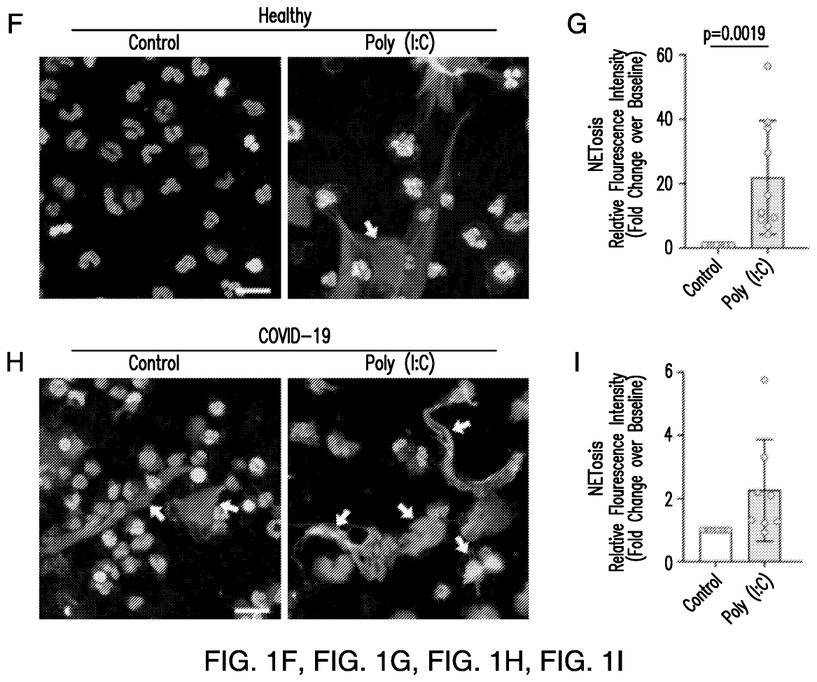

NETosis was assessed in autopsy lung specimens from three COVID-19 patients with fatal ARDS. Immunofluorescence revealed robust PMN infiltration based on MPO staining (FIG. 1A). Numerous citrullinated histone H3 positive PMNs were also observed, demonstrating that endogenous NETosis is upregulated in the human lung during COVID-19 (FIG. 1A). 23 COVID-19 patients and 14 age- and sex-matched healthy adults (Table 1) were enrolled and MPO-DNA complexes were measured to quantify plasma NETosis (one patient withdrew consent prior to blood draw). A significant increase in plasma NETosis was observed in non-intubated COVID-19 patients as well as endotracheally intubated COVID-19 patients compared to healthy donors (FIG. 1B). Plasma NETosis levels were significantly higher in COVID-19 non-survivors compared to COVID-19 survivors (FIG. 1C).

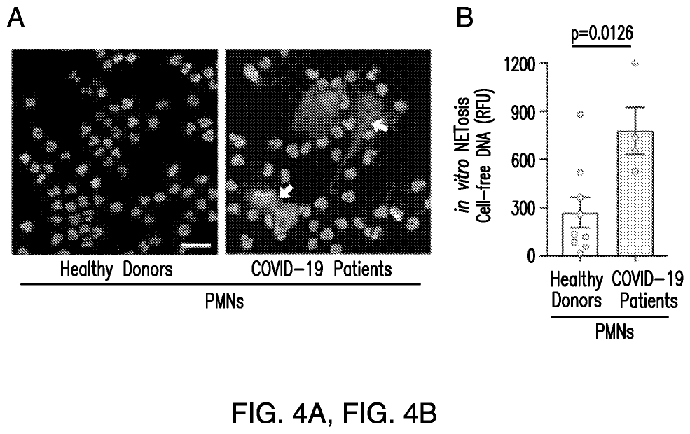

Next, whether plasma NETosis levels in COVID-19 patients correlate with PaO₂:FiO₂ ratio, a marker of respiratory failure, was determined. We found that PaO₂:FiO₂ ratios may inversely relate to NETosis (P=0.0505; FIG. 1D). NETosis levels were also significantly higher in tracheal aspirate fluid than in plasma samples in COVID-19 patients (FIG. 1E). Following recovery from COVID-19, plasma NETosis decreased to levels similar to that of healthy adults, indicating NETosis normalizes during convalescence after COVID-19 (FIG. 1B). Next, marked baseline NETosis levels in vitro in PMNs isolated from COVID-19 patients were demonstrated, while PMNs isolated from healthy adults remained quiescent under basal conditions (FIG. 4). Finally, it was demonstrated that poly (I:C), an RNA viral mimetic, induces NETosis in vitro by PMNs isolated from healthy adults as it does in preclinical models. A 2-fold increase in NETosis compared to the already elevated baseline levels when PMNs isolated from COVID-19 patients were stimulated with poly (I:C) were found (FIG. 1F).

1. NETosis Correlates with Microthrombi Formation and Platelet Deposition in COVID-19 Patients.

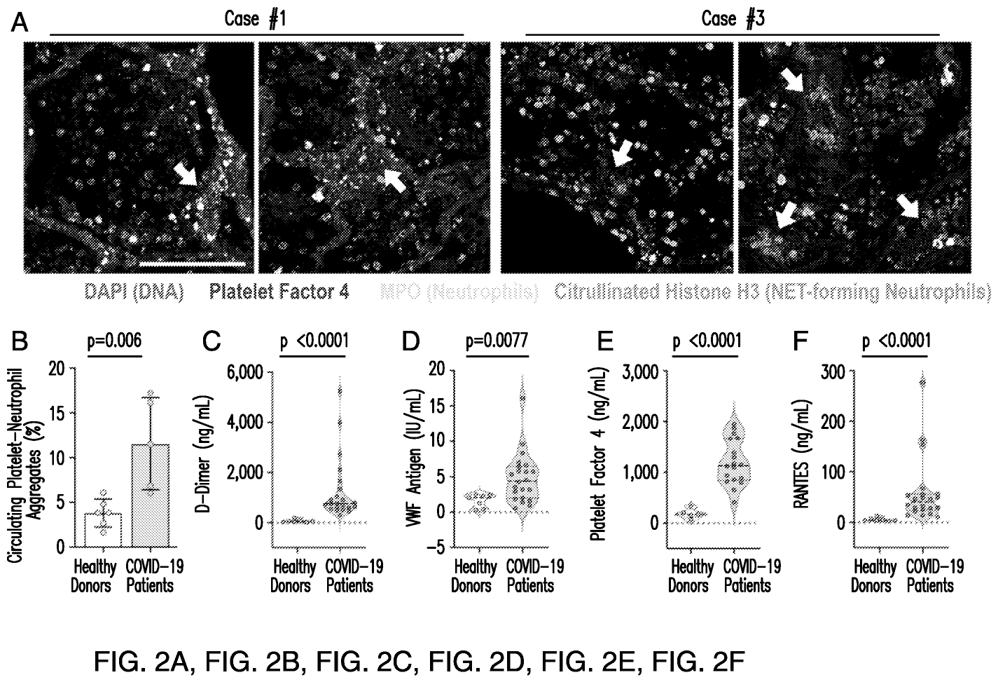

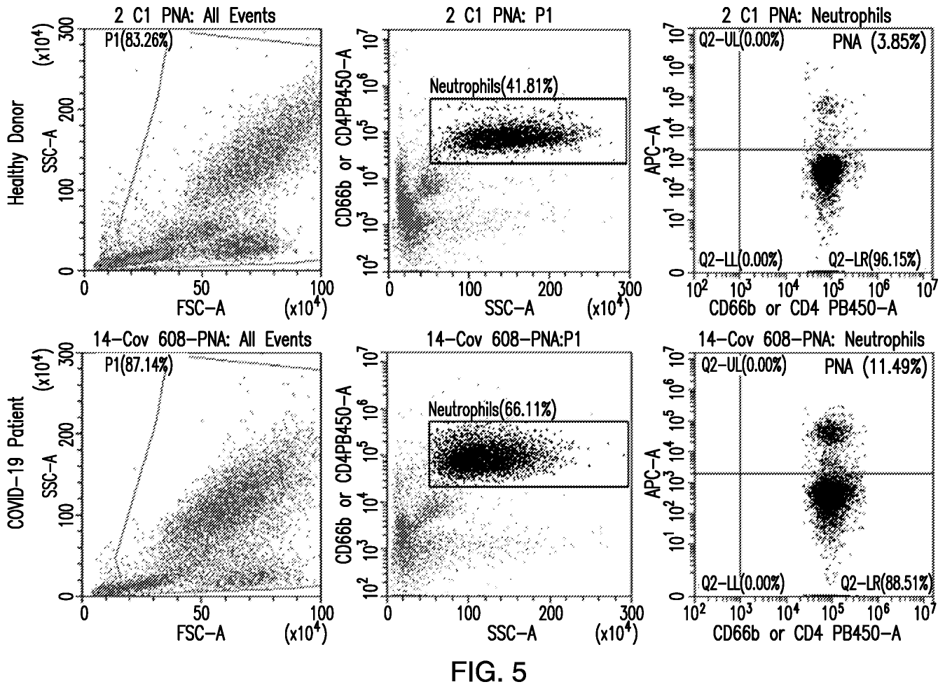

In additional studies of the lung autopsy samples, it was found that many of the neutrophils undergoing NETosis localize in blood vessels (FIGS. 1A, 2A). Using immunofluorescence, co-localization of NETosis with platelets (staining with PF4) was demonstrated in blood vessels of the lung (FIG. 2A), indicating that NETs trap platelets in COVID-19, a mechanism that can contribute to thrombosis in COVID-19 (FIG. 2A). Consistent with this, significantly higher levels of circulating platelet-neutrophil aggregates were detected in COVID-19 patients compared to healthy adults (FIG. 2B and FIG. 5). Soluble markers of thrombosis (e.g. plasma D-dimer levels and VWF), were also significantly elevated in COVID-19 patients (FIGS. 2C, D). Finally, elevated levels of soluble platelet activation markers that trigger NETosis, including PF4 and RANTES, were observed in COVID-19 patients (FIG. 2E, FIG. 2F). These data indicate that in COVID-19 patients, NETs trap activated platelets, thereby initiating a thrombo-inflammatory cascade contributing to hypercoagulability and thrombosis.

2. nNIF Blocks NETosis Induced by Soluble Factors in Plasma from COVID-19 Patients.

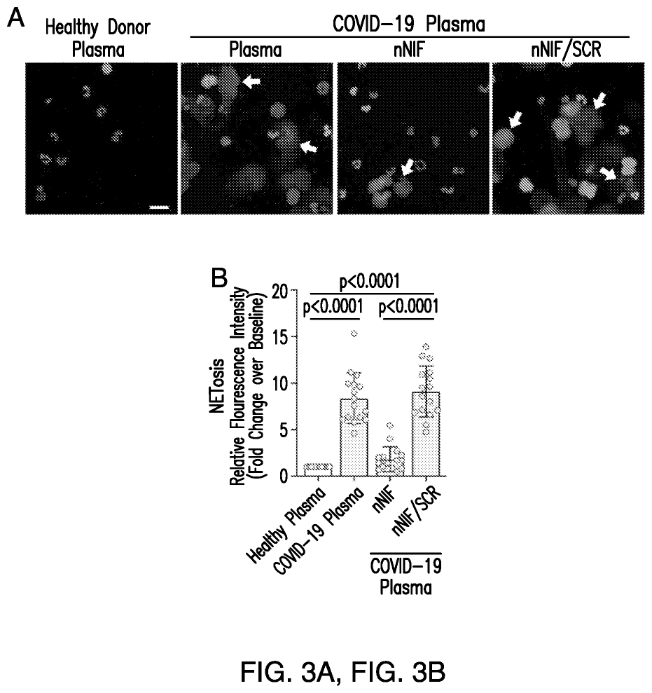

Incubation with plasma from COVID-19 patients induces NETosis by PMNs isolated from healthy adults, while plasma from healthy adults does not (FIG. 3). The analysis of COVID-19 plasma showed elevated levels of four NET-inducing factors compared to healthy adults: PF4 (FIG. 2E), RANTES (FIG. 2F), IL6 (0.3626±0.2055 v. 53.49±10.91 pg/mL; P=0.0017), and IL8 (4.236±1.280 v. 17.52±12.26 pg/mL; P=0.0058). Others have previously reported elevated plasma IL-6 and IL-8 levels in COVID-19 patients.

Given that an inhibitor of NETosis might reduce immunothrombosis, an approach to inhibit NETosis was assessed. Previously, a class of endogenous NET-inhibitory peptides (NIPs) was discovered circulating in human umbilical cord blood. These NET-inhibitory peptides are cleavage fragments of the carboxy-terminus of alpha-1-antitrypsin, a serine protease inhibitor. nNIF, a NET-inhibitory peptide which was synthesized based on the endogenous sequence, significantly decreases NETosis induced in vitro by COVID-19 plasma, while an inactive scrambled peptide control did not (FIG. 3).

### G. Discussion

In this cohort study, a robust association between NETosis, platelet activation, and severity of respiratory illness in COVID-19 is demonstrated. Our results support the use of plasma NETosis as a biomarker for COVID-19 patient outcomes. The endotracheally intubated COVID-19 patients with fatal ARDS displayed the highest levels of circulating NETosis, and lung specimens from three COVID-19 patients at autopsy demonstrated infiltration with NET-producing PMNs interacting with platelets. These data indicate that NETs contribute to COVID-19 related lung injury, similar to other reports of viral and autoimmune triggered NET-related ARDS. Along with a recent position paper and case series, these findings build upon and extend these prior findings by highlighting the potential importance of NETosis in the pathogenesis of COVID-19 related ARDS and its clinical sequelae. This study is the first to examine NETosis and thrombotic factors in a prospectively assembled cohort of COVID-19 patients, with plasma samples compared to age- and sex-matched controls. These findings indicate that NET-related immunothrombosis can offer insight into the unexplained observation of widespread microvascular and macrovascular thrombi in COVID-19 patients.

NETs were first described in 2004 and contribute to immunothrombosis, defined as a physiological form of thrombosis that enhances the innate immune response against pathogenic insults. Representing a primitive defense to microbial invaders, PMNs release NETs to physically capture and eliminate infections in the vasculature. The rapid release of NETs contributes to their maladaptive potential and the propagation of thrombi in the vasculature leading to multiorgan failure seen in systemic inflammatory syndromes. First recognized for their role in bacterial clearance, NETs are now firmly established as part of the innate defense against RNA-stranded viral pathogens, including RSV, Influenza, Dengue, and even HIV. Dysregulated NETosis triggered by respiratory viruses can lead to excessive acute lung injury.

The involvement of NETs in COVID-19 is supported by the well-described increase and recruitment of neutrophils in the pathophysiology and progression of SARS-CoV-2 pulmonary infection; neutrophil counts—the effector cell for NET production—increase with COVID-19 severity and ARDS. NET cytokine signatures have been demonstrated following SARS-CoV-2 infection in patient lungs (e.g., IL-1, TNF)34 and serum (e.g., IL-6).23 In this study, microvascular thrombi with neutrophils releasing NETs mixed with activated platelets were present in the lungs of COVID-19 patients, some with rapid disease progression. Increased baseline NETosis was found in PMNs tested in vitro from COVID-19 patients compared to healthy controls. Also reported were increased NETosis as measured by circulating MPO-DNA complexes in COVID-19 patient plasma that increases with disease severity, including high levels of NETosis in tracheal aspirates, and then returns to normal in convalescent patients, the first such observation (FIG. 1B). When coupled with an inverse relationship with oxygenation (PaO2/FiO2), these findings highlight a potential role for NETosis as a marker of increased disease severity and respiratory distress in COVID-19.

Similar to NETs, platelets play a role in the intravascular immune response and, if unchecked, contribute to inflammatory sequelae in sepsis. Platelets detect foreign pathogens through pattern recognition receptors, including the detection of viruses, and coordinate with PMNs to release NETs through chemokine and coagulation factor signaling (e.g., PF4, HMGB1, VWF). (Carestia A, Kaufman T, Schaffner M. Platelets: New Bricks in the Building of Neutrophil Extracellular Traps. Front Immunol 2016; 7:271) In the cohort, elevated levels of PF4 and platelet-neutrophil aggregates in COVID-19 patients were found. PF4, released from activated platelets, binds to NETs, making them compact and DNase-resistant, (Gollomp K, Kim M, Johnston I, et al. Neutrophil accumulation and NET release contribute to thrombosis in HIT. JCI Insight 2018; 3) and also can be released from platelets trapped inside thrombotic NETs. This PF4/NET loop thereby creates a coagulative NET cascade that might explain in part the high levels of PF4 and NETosis observed in COVID-19 patients.

One of the clinical observations in COVID-19 is the spectrum of thrombosis ranging from relatively asymptomatic patients with sudden ischemic cerebral events and myocardial infarctions to ICU patients on ventilators with disseminated intravascular coagulopathy, stroke, venous embolism, and multiorgan failure. In COVID-19, NETs and activated platelets increase the risk for thrombosis with potential pathologic sequelae. This report shows high levels of markers associated with thrombosis including VWF, D-dimers, and PF4. Considered together with the increasing levels of plasma MPO-DNA complexes indicating NETs, the clinical manifestations of severe COVID-19 can be explained by immunothrombosis and NETosis in response to SARS-CoV-2.

Inhibition of NETosis represents an enticing target for clinical intervention in this COVID-19 pandemic and clinical trials are rapidly being conceptualized to target dysregulated NETosis in COVID-19 patients. Here evidence is presented that synthesized nNIF, an inhibitor of NETosis discovered in human umbilical cord blood, inhibits NETs induced by COVID-19 plasma in PMNs from healthy adults. Preclinical testing of nNIF in models of experimental sepsis demonstrates decreased disease severity and improved survival with an accompanying decrease in NETosis. nNIF and related NET-inhibitory peptides are now being evaluated in the preclinical setting for their therapeutic potential in COVID-19 and other diseases associated with immunothrombosis.

### Example 2

Elevated NETosis biomarkers have been determined in COVID-19 patient plasma. Plasma samples were collected from healthy volunteers or COVID-19 patients from 2 centers in Israel. Complexes of MPO-DNA were measured as surrogate markers for NETs in plasma. Patients with mild-moderate or severe/critical COVID-19 had elevated MPO-DNA as compared to healthy controls or patients who had recovered from COVID-19 (convalescence samples). Notably, the levels of MPO-DNA complexes, although reduced compared to acute disease, are still elevated in convalescent patients.

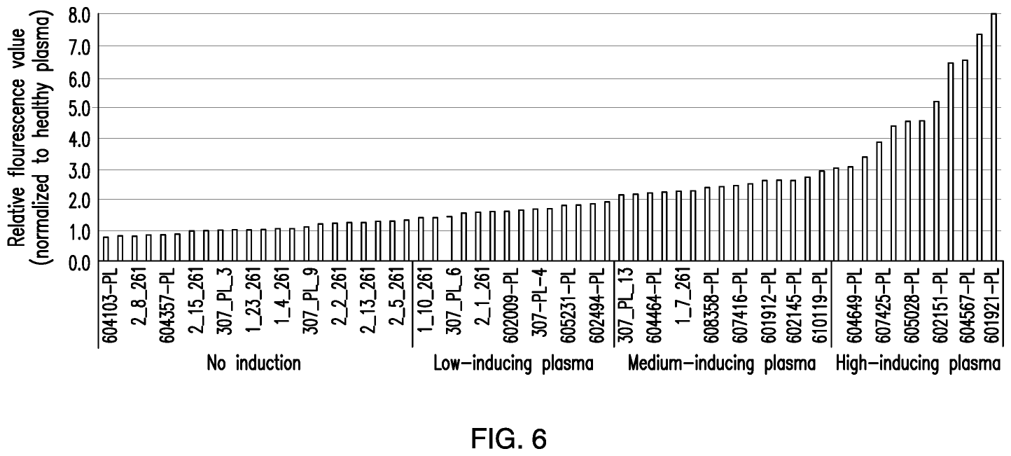

Differential NETosis induction by COVID-19 patient plasma was determined in plasma samples screened for NET-inducing ability in healthy neutrophils, and sorted by induction level. Normal neutrophils isolated from healthy donors release NETs upon exposure to COVID-19 patient plasma. The level of NETosis induction by different patient plasma is, however, variant. FIG. 6 represents the sorting of about 60 patient plasmas, based on their level over healthy-neutrophil-NETosis-induction (value set to 1; low=1.2-2, medium=2-3, high >3-fold increase). The induction level is measured using SYTOX Green, which in these experimental settings will only measure extracellular DNA.

A correlation has been shown between NETosis induction and platelet factors, which points to the NETosis induction mechanism of action being platelet-mediated. The variation in level of induction can be correlated with the presence of two cytokines indicative of systemic platelet activation: RANTES and platelet factor 4.

A correlation has been shown between NETosis induction and endothelial cell/platelet factors which points to the NETosis induction mechanism of action being platelet-mediated with potential contribution from activated endothelial cells. The variation in level of induction can also be correlated with the amount of soluble P-selectin.

A correlation has been shown between NETosis induction and platelet factors which points to the NETosis induction mechanism of action being platelet-mediated with potential contribution from activated endothelial cells. The variation in level of induction can also be correlated with the amount of von willibrand factor (VWF).

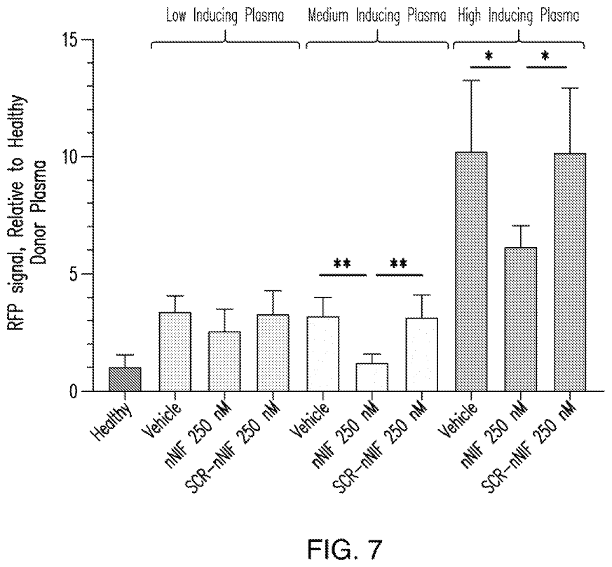

FIG. 7 shows ex vivo results showing NIP (e.g. nNIF) inhibition of COVID-19 plasma induced NETosis. nNIF can inhibit NET formation in COVID-19-plasma induced NET assay of both high and medium-inducing potential. Plasmas from COVID-19 patients screened for NET-inducing capability were sorted by induction level and pooled together based on similar NET-inducing potential to three stocks: low, medium, or high-inducing. The stocks were incubated with healthy neutrophils pretreated with vehicle, nNIF or nNIF-SCR (SCR=scrambled). Inhibition of COVID19 plasma induced NETosis by nNIF, is clearly observed in medium and high-inducing plasma conditions.

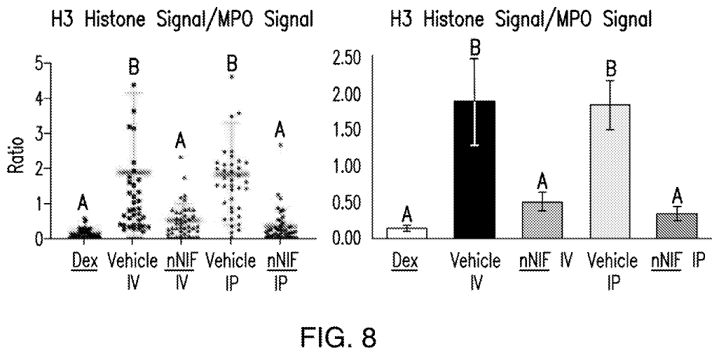

FIG. 8 shows a comparison of treatment with dexamethasone and nNIF. The data show the in vivo efficacy of both dexamethasone and nNIF in a LPS-induced acute lung inflammation model. Cytospin results show very good efficacy with IP administration of nNIF similar to dexamethasone treatment (less NETs or cells undergoing early NETosis). Thus, nNIF can reduce NETs similar to an FDA approved drug, such as dexamethasone.

Those skilled in the art will recognize, or be able to ascertain using no more than routine experimentation, many equivalents to the specific embodiments of the method and compositions described herein. Such equivalents are intended to be encompassed by the following claims.

We claim:

1. A method of treating neutrophil extracellular trap (NET) mediated inflammatory tissue damage and/or NET mediated microthrombi in a patient infected with SARS-CoV-2, comprising:
administering to the patient infected with SARS-CoV-2 an effective amount of a pharmaceutical composition comprising a NET-inhibitory peptide (NIP) and a pharmaceutically acceptable carrier to reduce a pathological effect or symptom of the NET-mediated inflammatory tissue damage and/or to reduce NET mediated microthrombi in the patient.

2. The method of claim 1, wherein the NIP is a neonatal NET-Inhibitory Factor (nNIF) or nNIF Related Peptide (NRP).

3. The method of claims 1-2, wherein the NIP is a peptide comprising the amino acid sequence of SEQ ID NO:1, SEQ ID NO:2 or SEQ ID NO:3 and a pharmaceutically acceptable carrier to reduce a pathological effect or symptom of the NET-mediated inflammatory tissue damage.

4. The method of claims 1-3, wherein the NIP is a peptide consisting of the amino acid sequence of SEQ ID NO:1, SEQ ID NO:2 or SEQ ID NO:3 and a pharmaceutically acceptable carrier to reduce a pathological effect or symptom of the NET-mediated inflammatory tissue damage.

5. The method of any one of claims 1-4, wherein NET mediated inflammatory tissue damage and/or NET mediated microthrombi is reduced in an effective amount.

6. The method of any one of claims 1-5, wherein NET mediated inflammatory tissue damage and/or NET mediated microthrombi is reduced by 5-50%.

7. The method of any one of claims 1-6-4, wherein NET mediated inflammatory tissue damage and/or NET mediated microthrombi is reduced by 5-50% when compared to a sample from the patient infected with SARS-CoV-2 that did not receive an effective amount of the pharmaceutical composition comprising a NET-inhibitory peptide (NIP) and a pharmaceutically acceptable carrier.

8. A method of inhibiting neutrophil extracellular trap (NET) formation in a patient infected with SARS-CoV-2 comprising:
administering to the patient infected with SARS-CoV-2 an effective amount of a pharmaceutical composition comprising a NET-inhibitory peptide (NIP) and a pharmaceutically acceptable carrier to substantially inhibit NET formation.

9. The method of claim 8, wherein the NIP is a nNIF or NRP.

10. The method of claims 8-9, wherein the NIP is a peptide comprising the amino acid sequence of SEQ ID NO:1, SEQ ID NO:2 or SEQ ID NO:3 and a pharmaceutically acceptable carrier to substantially inhibit NET formation.

11. The method of claims 8-10, wherein the NIP is a peptide consisting of the amino acid sequence of SEQ ID NO:1, SEQ ID NO:2 or SEQ ID NO:3 and a pharmaceutically acceptable carrier to substantially inhibit NET formation.

12. The method of any one of claims 8-11, wherein NET formation is inhibited in an effective amount.

13. The method of any one of claims 8-12, wherein NET formation is inhibited by 5-50%.

14. The method of any one of claims 8-13, wherein NET formation is inhibited by 5-50% when compared to a sample from the patient infected with SARS-CoV-2 that did not receive an effective amount of the pharmaceutical composition comprising a NET-inhibitory peptide (NIP) and a pharmaceutically acceptable carrier.

15. A method of inhibiting NETosis induced by soluble factors in plasma in a patient infected with SARS-CoV-2 comprising:
administering to the patient infected with SARS-CoV-2 an effective amount of a pharmaceutical composition comprising a NET-inhibitory peptide (NIP) and a pharmaceutically acceptable carrier thereby inhibiting NETosis induced by soluble factors in plasma in a patient infected with SARS-CoV-2.

16. The method of claim 15, wherein the NIP is a neonatal NET-Inhibitory Factor (nNIF) or nNIF Related Peptide (NRP).

17. The method of claims 15-16, wherein the NIP is a peptide comprising the amino acid sequence of SEQ ID NO:1, SEQ ID NO:2 or SEQ ID NO:3 and a pharmaceutically acceptable carrier to inhibit NETosis induced by soluble factors in plasma in a patient having COVID-19.

18. The method of any one of claims 15-17, wherein NETosis induced by soluble factors in plasma is inhibited by 5-50%.

19. The method of claim 18, wherein NETosis induced by soluble factors in plasma is inhibited by 5-50% when compared to a sample from the patient infected with SARS-CoV-2 that did not receive an effective amount of the pharmaceutical composition comprising a NET-inhibitory peptide (NIP) and a pharmaceutically acceptable carrier.

20. A method of preventing neutrophil extracellular trap (NET) mediated inflammatory tissue damage and/or NET mediated microthrombi in a patient infected with SARS-CoV-2, comprising:
administering to the patient infected with SARS-CoV-2 an effective amount of a pharmaceutical composition comprising a NET-inhibitory peptide (NIP) and a pharmaceutically acceptable carrier to reduce a pathological effect or symptom of the NET-mediated inflammatory tissue damage and/or to reduce NET mediated microthrombi.

21. A method of preventing plasma from a SARS-CoV-2 infected patient from inducing NET formation in the patient comprising:
administering to a patient an effective amount of a pharmaceutical composition comprising a NET-inhibitory peptide (NIP) and a pharmaceutically acceptable carrier to reduce NET formation from neutrophils induced by the plasma.

22. The method of any of claims 1-21, wherein the patient has medium or high inducing plasma.

23. The method of claim 6, wherein the patient has medium or high inducing plasma and the NET mediated inflammatory tissue damage and/or NET mediated microthrombi is reduced by 5-20% as compared to a patient with low inducing plasma.

24. The method of claim 13, wherein the patient has medium or high inducing plasma and the wherein NET formation is inhibited by 5-20% as compared to a patient with low inducing plasma.

25. The method of claim 18, wherein the patient has medium or high inducing plasma and NETosis induced by soluble factors in plasma is inhibited by 5-20% as compared to a patient with low inducing plasma.

26. A method of reducing one or more platelet activation factors in a patient infected with SARS-CoV-2 comprising:
administering to the patient infected with SARS-CoV-2 an effective amount of a pharmaceutical composition comprising a NET-inhibitory peptide (NIP) and a pharmaceutically acceptable carrier to substantially reduce the one or more platelet activation factors.

27. The method of claim 26, wherein the one or more platelet activation factors are RANTES, platelet factor 4, von willibrand factor, or P-selectin.

28. The method of any of claims 1-27, wherein prior to administering the effective amount of a pharmaceutical composition comprising a NET-inhibitory peptide (NIP) and a pharmaceutically acceptable carrier, the patient is identified as having medium or high inducing plasma.

29. The methods of any one of claims 1-268, further comprising administering one or more known standards of care for coronavirus infection.

30. The method of claim 299, wherein the one or more known standards of care for coronavirus infection are antibodies, nanobodies, antiviral small molecules, macromolecules of sulfated polysaccharides, or polypeptides.
